(19) United States
(12) Patent Application Publication
(10) Pub. No.: US 2012/0189688 A1
(43) Pub. Date: Jul. 26, 2012

(54) CARRIER COMPONENT AND FABRICATION METHOD THEREOF

(75) Inventors: Dean-Mo Liu, Hsinchu (TW); Chun Yu Chang, Hsinchu (TW); Fu Hsuan Chou, Hsinchu (TW); Tin-Yo Yen, Vancouver (CA)

(73) Assignee: NATIONAL CHIAO TUNG UNIVERSITY, Hsinchu City (TW)

(21) Appl. No.: 13/112,170

(22) Filed: May 20, 2011

(30) Foreign Application Priority Data
Jan. 21, 2011 (TW) 100102234

(51) Int. Cl. A61K 9/127; A61K 38/28; A61K 31/713; A61K 31/7088; A61K 38/02; A61P 25/08; A61P 9/12; A61P 3/10; A61P 31/10; A61P 37/08; A61P 31/00; A61K 9/00; A61P 35/00; B82Y 5/00

(52) U.S. Cl. 424/450; 424/400; 514/5.9; 514/44 A; 514/1.1; 977/773; 977/810; 977/838; 977/906; 977/907

(57) ABSTRACT

The present invention provides a carrier component. The carrier component includes a carrier core body including a dispersive object and a dualistic self-assembly material for encapsulating the dispersive object, wherein the dualistic self-assembly material has an electric charge; and a first shell layer having an electric charge opposite to the electric charge of the dualistic self-assembly material, and coating the carrier core body, and thus avoids inactivation of a medicine, eliminates medicine leakage and reduces medicine releasing. The present invention further provides a method for forming a carrier component.

### BACKGROUND OF THE INVENTION

1. Field of the Invention

The present invention relates to a carrier component, and more particularly, to a carrier for a nano-medicine and a fabrication method thereof.

2. Description of Related Art

A carrier for a nano-medicine is used for carrying a medicine at molecular level, and for treating diseases, which are difficult to be treated in the conventional art. It is the trend to develop a carrier for a nano-medicine in the medical field.

In the conventional carrier for a nano-medicine, a hallow nano-ball body is formed by a layer-by-layer treatment, acid cleaning or calcining. However, the calcination with high temperature or acid cleaning results in disrupting activity of the medicine, and thus reduces therapeutic effect.

In addition, oily medicines are coated with self-assembly material having hydrophilic groups and hydrophobic groups to form nanomicelles in the medical field. Some conventional carriers are also prepared by the self-assembly technology. However, the medicine carrier formed by such technology generally results in medicine leakage due to the swelling of molecules with high molecular weight.

U.S. Pat. No. 7,744,644 discloses a drug delivery device having a surface with a layer-by-layer structure, wherein there is static electricity among layers. However, the size of such device is too big to be used in nano medical field such as gene therapy. Further, the layer-by-layer structure is formed on a flat substrate, which restricts the coating of medicines and results in medicine leakage.

U.S. Pat. No. 7,758,892 discloses a drug delivery device, which has multiple-layer structure formed on a flat substrate and thus also results in medicine leakage.

U.S. Pat. No. 7,763,275 discloses a method for forming nanocapsules and forming a layer-by-layer structure surrounding a template without cleaning, separation or filtration. However, this method still needs calcination or acid cleaning to remove a mold, so as to obtain nanocapsules, and therefore the activity of the medicine is reduced.

Chinese Patent No. 1660082 discloses a microcapsule and a method for forming the same, in which a layer-by-layer structure is formed on the surface of a micro-crystal. However, the medicine is coated by the layer-by-layer structure, and thus easily results in medicine leakage due to the swelling of molecules with high molecular weight.

Therefore, there is a need to develop a carrier for a nano-medicine, which is capable of preventing inactivation of medicines, decreasing medicine leakage and reducing medicine releasing rate.

### SUMMARY OF THE INVENTION

The present invention provides a carrier component, including a carrier core body including a dispersive object and a dualistic self-assembly material for encapsulating the dispersive object, wherein the dualistic self-assembly material has an electric charge; and a first shell layer having an electric charge opposite to the electric charge of the dualistic self-assembly material, and encapsulating the carrier core body.

In addition, the present invention also provides a method for forming a carrier component, including the steps of: (A) dissolving a dispersive object in a solution containing a dualistic self-assembly material to form a carrier core body including the dispersive object and the dualistic self-assembly material encapsulating the dispersive object; and (B) providing in the solution a first molecule having an electric charge opposite to an electric charge of the dualistic self-assembly material to form a first shell layer for encapslating the carrier core body, so as to form the carrier component.

Accordingly, carrier component and the method for forming the carrier component use a layer-by-layer structure and self-assembly technology at room temperature or lower temperature without acid cleaning and calcination, so as to avoid disruption of activity of the carrier object such as a medicine. Therefore, the present invention provides a carrier for a nano-medicine which prevents inactivation of the medicine, eliminates medicine leakage and reduces medicine releasing rate.

### BRIEF DESCRIPTION OF THE DRAWINGS

FIGS. 1A to 1F are schematic views showing the method for forming the carrier component according to the present invention, in which FIG. 1E and FIG. 1F are schematic views showing the carrier component having the interlayered medicine;

FIG. 2 is a diagram showing the potential analysis at each step in Embodiment 1 of the present invention;

FIGS. 3A to 3C show SEM diagrams at each step of forming the carrier component of the present invention, in which FIG. 3A is the SEM diagram showing the carrier core body, FIG. 3B is the SEM diagram showing the first shell layer of the carrier component, and FIG. 3C is the SEM diagram showing the second shell layer of the carrier component; and

FIG. 4 shows analysis results of medicine releasing rate of the carrier core body and the carrier component having the first shell layer according to Embodiment 1 of the present invention; and

FIG. 5 shows the analysis result of the medicine releasing rate of the carrier component according to the embodiment of the present invention.

### DETAILED DESCRIPTION OF THE PREFERRED EMBODIMENTS

The detailed description of the present invention is illustrated by the following specific examples. Persons skilled in the art can conceive the other advantages and effects of the present invention based on the disclosure contained in the specification of the present invention.

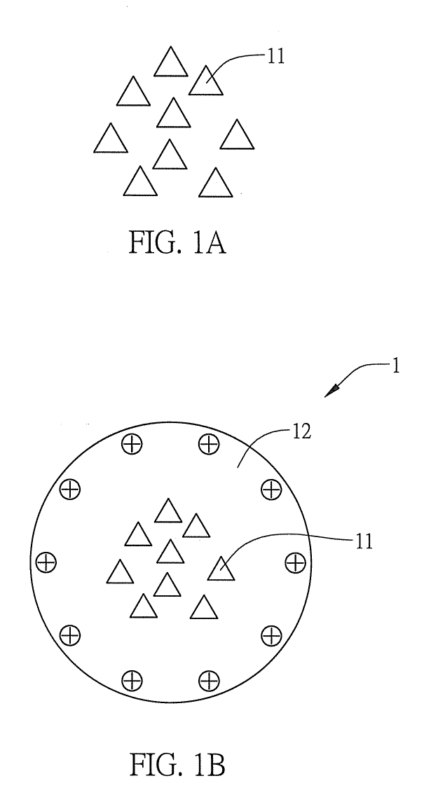

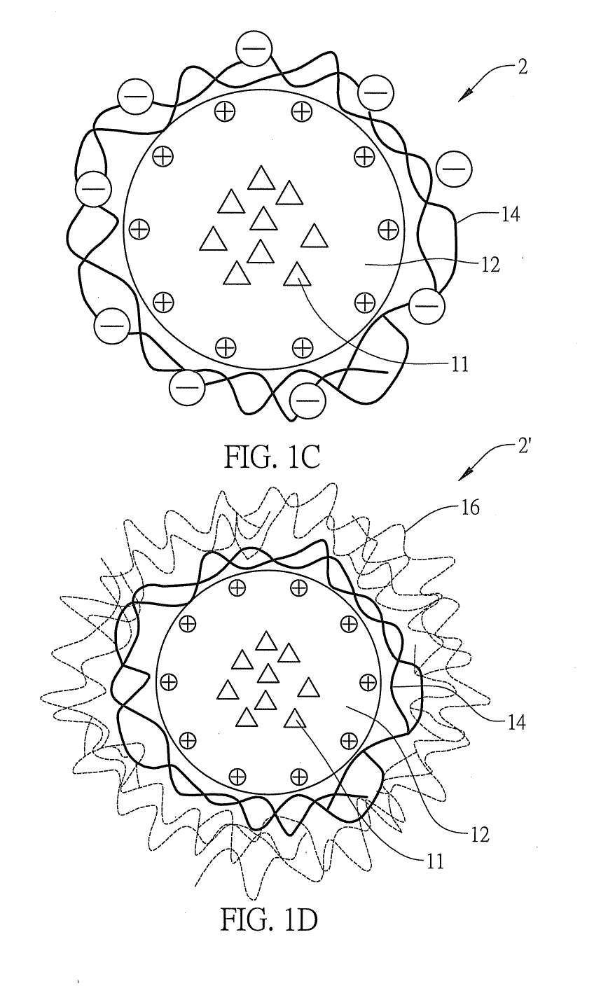

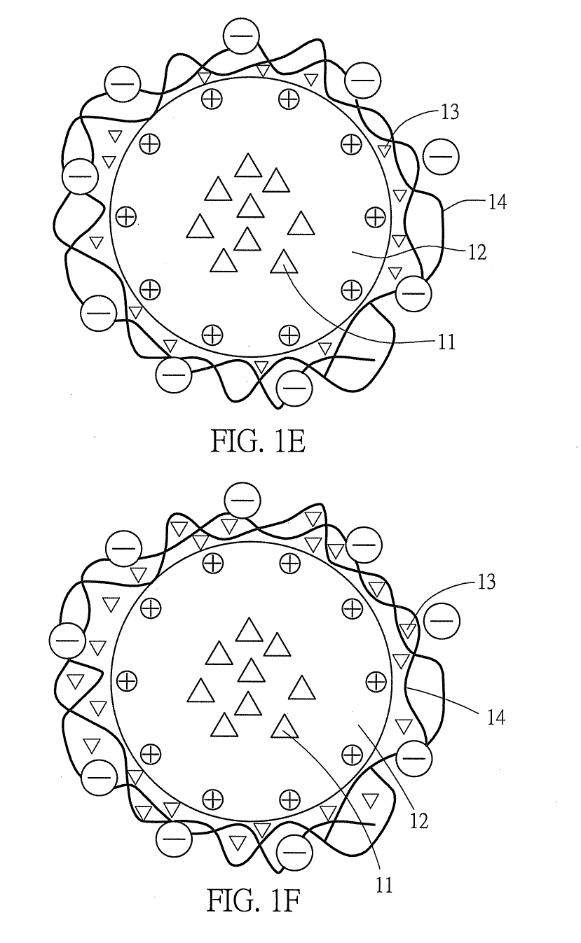

FIGS. 1A to 1D are schematic views showing the method for forming the carrier component according to the present invention. The method of the present invention includes the steps (A) and (B).

(A) As shown in FIG. 1A and FIG. 1B, dispersive objects 11 are dissolved in solution containing dualistic self-assembly material 12 to form a carrier core body 1 including the dispersive objects 11 and the dualistic self-assembly material 12 the dispersive objects 11. The “self-assembly material” herein refers to the material which forms aggregates of nano-micelles or hallow nano-capsules. Further, the term “dualistic” herein refers to the material which is both hydrophilic and hydrophobic, and thus forms aggregates of nano-micelles and hollow nano-capsules.

In the method of the present invention, the dualistic self-assembly material is an amphipathic chitosan, an amphipathic gel, a micro liposome or poly (lactic-co-glycolic acid). Preferably, the dualistic self-assembly material is an amphipathic chitosan. Specifically, the dualistic chitosan in the solution coats the dispersive or dissolved dispersive objects by self-assembly, so as to form the carrier core body 1. The amphipathic chitosan is both hydrophilic and hydrophobic, and thus forms aggregates of nano-micelles in the solution via the interaction of hydrophilic groups and hydrophobic groups, in which the dispersive objects are coated to form the carrier core body 1.

In the previous step, the dispersive object may be a fluorescent molecule, a hydrophilic medicine, a hydrophobic medicine, a hydrophilic and hydrophobic medicine or a biological molecule. Preferably, the dispersive object is a hydrophilic and hydrophobic medicine.

The dispersive object may be a medicine selected from the group consisting of an anticancer drug, an anti-differentiation drug, an anti-hypertension drug, an anti-microbial drug, an anti-diabetes drug, an anti-fungal drug, an antiepilepsy drug, an anti-allergy drug, an SiRNA, a miRNA, a peptide, a protein, an insulin, or a derivative thereof.

(B) As shown in FIG. 1C, a first molecule having an electric charge opposite to the electric charge of the dualistic self-assembly material 12 is added into the solution to form a first shell layer 14 for encapsulating the carrier core body 1, and thus obtain the carrier component 2. The first molecule forming the first shell layer 14 may be poly(sodium styrenesulfonate), poly(acrylic acid) (PAA), polycyclic aromatic hydrocarbon (PAH), poly (1,4-phenylene vinylene), silicon oxide or a nano gold particle. Preferably, the first molecule is poly(sodium styrenesulfonate). In one embodiment, the dualistic self-assembly material is a amphipathic chitosan with positive charges, and the first molecule is poly(sodium styrenesulfonate) with negative charges, such that the first shell layer 14 is formed via electrostatic force.

As shown in FIG. 1D, the method for forming a carrier component in the present invention further includes the step (C), in which a second molecule having an electric charge opposite to the electric charge of the first shell layer 14 is added to form a second shell layer 16. In one embodiment, the second molecule is a hydrophilic self-assembly material, such as a hydrophilic chitosan.

In one embodiment, the method further includes a step (B-1) between the step (B) and the step (C), wherein the first molecule which is not absorbed on the surface of the first shell layer 14 is removed. In another embodiment, the method of the present invention further includes the step (C-1) after the step (C), wherein the carrier component 2′ is cleaned with deionized water.

In addition, in one embodiment of the method, the step (B) and the step (C) are repeated for at least once, so as to form a layer-by-layer structure of alternate first shell layers 14 and second shell layers 16. Therefore, the encapsulation rate of the carrier component is increased. In one embodiment, the carrier component is a carrier component for a medicine, and the encapsulation rate of the carrier component is 90 to 100%. The encapsulation rate is measured by a UV/visible spectrophotometer or high-performance liquid chromatograpgy (HPLC), and the calculation equation is as follows.

$$EE - \frac{{D}_{total} - {D}_{free}}{{D}_{total}} \times 100 \%$$

EE: encapsulation rate; Dₜₒₜₐₗ: initial drug amount in solution; Dₜₒₜₐₗ: uncoated free drug in solution

In one embodiment, the method of the present invention may further include the step (A-1) between the step (A) and the step (B), wherein an interlayered medicine 13 is added in the solution to deposit the interlayered medicine 13 in the step (A-1) and the first shell layer 14 in the step (B) in sequence, such that the interlayered medicine 13 is disposed between the carrier core body 1 and the first shell layer 14 as shown in FIG. 1E. The interlayered medicine 13 has no electric charge or an electric charge opposite to the electric charge of the dualistic self-assembly material 13. Preferably, the interlayered medicine 13 has an electric charge opposite to the electric charge of the dualistic self-assembly material 13.

Further, in one embodiment, the interlayered medicine 13 and the first molecule are co-deposited on the surface of the carrier core body 1, and the interlayered medicine 13 is disposed in the first shell layer 14. Specifically, the solution in the step (B) further includes the interlayered medicine 13, and thus the interlayered medicine 13 is buried in the first shell layer 14 as shown in FIG. 1F. The method of the present invention may be performed at room temperature or at lower temperature, and there is no need to perform acid cleaning and calcination, so as to avoid disruption of the objects such as the activity of the medicine. In the method of the present invention, the temperature of the solution is in a range from −100 to 100° C., preferably in a range from 20 to 100° C., and more preferably in a range from 25 to 100° C.

According to the method of the present invention, the present invention provides a carrier component 2, 2′ as shown in FIG. 1C and FIG. 1D. The carrier component includes a carrier core body 1 having a dispersive object 11 and a dualistic self-assembly material 12 coating the dispersive object 11, wherein the dualistic self-assembly material 11 has an electric charge; and a first shell layer 14 having an electric charge opposite to the electric charge of the dualistic self-assembly material 12 and encapsulating the carrier core body 1.

The carrier component of the present invention further includes a second shell layer 16 having an electric charge opposite to the electric charge of the first shell layer 14. In one embodiment, the carrier component of the present invention further includes a layer-by-layer structure formed of multiple first shell layers 14 and multiple second shell layers 16.

The term “layer-by-layer structure” herein refers to alternate stacks of the first shell layer 14 and the second shell layer 16. In addition, the absorption between the first shell layer 14 and the second shell layer 16 is via electrostatic force.

In the carrier component of the present invention, the dualistic self-assembly material may be an amphipathic chitosan, an amphipathic gel, a micro liposome or poly (lactic-co-glycolic acid). Preferably, the dualistic self-assembly material is an amphipathic chitosan. Specifically, the dualistic chitosan in the solution coats the dispersive or dissolved dispersive objects by self-assembly, so as to form the carrier core body 1. The amphipathic chitosan is both hydrophilic and hydrophobic, and thus forms aggregates of nano-micelles in the solution via the interaction of hydrophilic groups and hydrophobic groups, in which the dispersive objects 11 are coated to form the carrier core body 1.

In one embodiment, the dualistic chitosan is a chitosan derivative of formula (I):

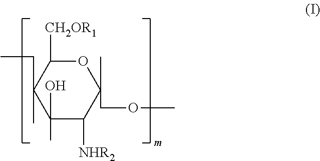

wherein R₁ is independently hydrogen, C₁-C₄alkyl, C₁-C₆carboxyl, sulfate or phosphate, R₂ is independently hydrogen, C₁-C₁₂alkyl, C₁-C₆carboxyl or C₂-C₁₂acyl, and m is an integer from 100 to 2000.

In another embodiment, the dualistic chitosan is a chitosan derivative of formula (I):

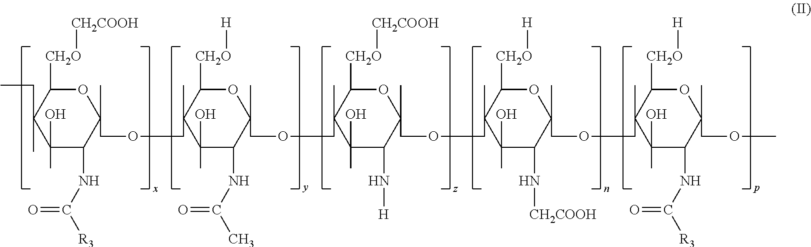

wherein R₃ is independently C₅-C₁₁alkyl, and x, y, z, n and p are integers independently from 20 to 2000.

The dispersive object may be a fluorescent molecule, a hydrophilic medicine, a hydrophobic medicine, a hydrophilic and hydrophobic medicine or a biological molecule. Preferably, the dispersive object is a hydrophilic and hydrophobic medicine.

Further, in the carrier component of the present invention, the dispersive object may be a medicine selected from the group consisting of an anticancer drug, an anti-differentiation drug, an anti-hypertension drug, an anti-microbial drug, an anti-diabetes drug, an anti-fungal drug, an antiepilepsy drug, an anti-allergy drug, an SiRNA, an miRNA, a peptide, a protein, an insulin, or a derivative thereof.

In the carrier component of the present invention, the first shell layer 14 may be made of poly(sodium styrenesulfonate), poly(acrylic acid) (PAA), polycyclic aromatic hydrocarbon (PAH), poly (1,4-phenylene vinylene), silicon oxide or a nano gold particle. Preferably, the first shell layer 14 is made of poly(sodium styrenesulfonate). In one embodiment, the second shell layer 16 is a hydrophilic self-assembly material such as a hydrophilic chitosan.

In the carrier component of the present invention, the diameter of the carrier core body 1 is preferably in a range from 50 to 300 nm, and more preferably in a range from 100 to 150 nm. The diameter of the carrier component is preferably in a range from 200 to 500 nm, and more preferably in a range from 200 to 250 nm. In one embodiment, the diameter of the carrier core body is in a range from 100 to 150 nm, and the diameter of the carrier component is in a range from 200 to 250 nm.

In the carrier component of the present invention, the dispersive object is a fluorescent molecule, a hydrophilic medicine, a hydrophobic medicine, a hydrophilic and hydrophobic medicine or a biological molecule. Preferably, the dispersive object is a hydrophilic and hydrophobic medicine.

The carrier component may be a carrier component for a medicine, and the encapsulation rate of the carrier component is 90 to 100%. In one embodiment, the dispersive object may be a medicine selected from the group consisting of an anticancer drug, an anti-differentiation drug, an anti-hypertension drug, an anti-microbial drug, an anti-diabetes drug, an anti-fungal drug, an antiepilepsy drug, an anti-allergy drug, an SiRNA, an miRNA, a peptide, a protein, an insulin, or a derivative thereof.

In another embodiment, the carrier component of the present invention further includes at least an interlayered object disposed between the carrier core body 1 and the first shell layer 14 (as shown in FIG. 1E), in the first shell layer 14 (as shown in FIG. 1F), between the first shell layer 14 and the second shell layer 16, or in the second shell layer 16. For example, the interlayered object 13 may be an interlayered medicine or developing material. When the dispersive object and the interlayered object are medicines, the interlayered medicine and the dispersive object may be different medicines. The interlayered medicine is one selected from the group consisting of an anticancer drug, an anti-proliferative drug, an anti-hypertension drug, an anti-microbial drug, an anti-diabetes drug, an anti-fungal drug, an antiepilepsy drug, an anti-allergy drug, an SiRNA, an miRNA, a peptide, a protein, an insulin, or a derivative thereof. The developing material is ferric oxide, gadolinium oxide, gadolinium complex, a platinum particle or a gold particle.

### Embodiment 1

An anticancer drug (S)-(+)-camptothecin (CPT) 11 was used as a dispersive object, and had the concentration of 100 ug/mL.

The modification of the dualistic chitosan was performed. 5 g of chitosan (Mw=215,000 g/mol, deacetylation=80-90%, purchased from Adrich-Sigma) was suspended in isopropanol (50 mL), and stirred for 30 minutes. The suspension solution was slowly mixed with NaOH solution (12.5 mL) to form a mixed solution. The NaOH concentration of the mixed solution was adjusted to 13.3 M. The reaction of the mixed solution and chloroacetic acid was performed to form hydrophilic carboxymethyl-modified chitosan, which was then dried.

2 g of the dried hydrophilic carboxymethyl-modified chitosan was dissolved in pure water (50 mL), and stirred for 24 hours. The solution was then mixed with methanol (50 mL) and added with hexanoic acid anhydride (0.2 M) to form a reaction solution. The reaction in the reaction solution was performed for 20 hours, and the reaction solution was then collected and dialyzed with ethanol solution (25% v/v) for 24 hours. The product was dried to obtain a dualistic chitosan modified with the carboxylmethyl group and the hexanoyl group and having a hydrophilic end and a hydrophobic end.

The modified dualistic chitosan was dissolved to form 2% aqueous solution.

Then, the CPT 11 was added into the dualistic chitosan solution. After the self-assembly was performed for 8 hours, the dualistic chitosan was self-assembled to form a carrier core body coating the CPT 11.

The carrier core body solution was adjusted to pH 6.5 and then added with poly(sodium styrenesulfonate) solution with negative charges, which has the identical volume and concentration to those of the modified dualistic chitosan, in which the carrier core body was covered with poly(sodium styrenesulfonate). The mixture was cleaned with deionized water to remove poly(sodium styrenesulfonate), which was not absorbed on the surface of the carrier core body. The poly(sodium styrenesulfonate) absorbed on the carrier core body was the first shell layer.

An aqueous solution was added with the hydrophilic chitosan solution, which has the volume and concentration identical to those of the modified dualistic chitosan solution, so as to form the carrier component having the second shell layer due to electrostatic force between the hydrophilic chitosan and poly(sodium styrenesulfonate). The mixture was cleaned with deionized water to remove residual hydrophilic chitosan.

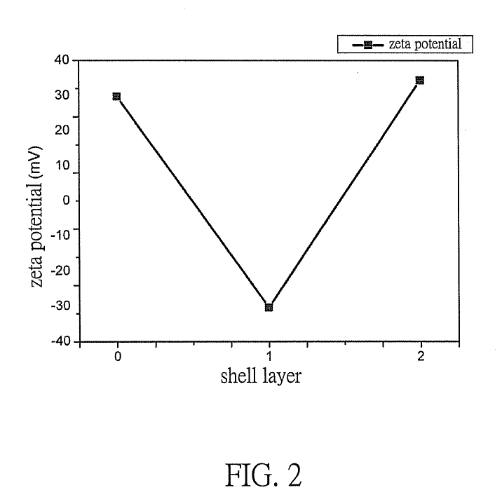

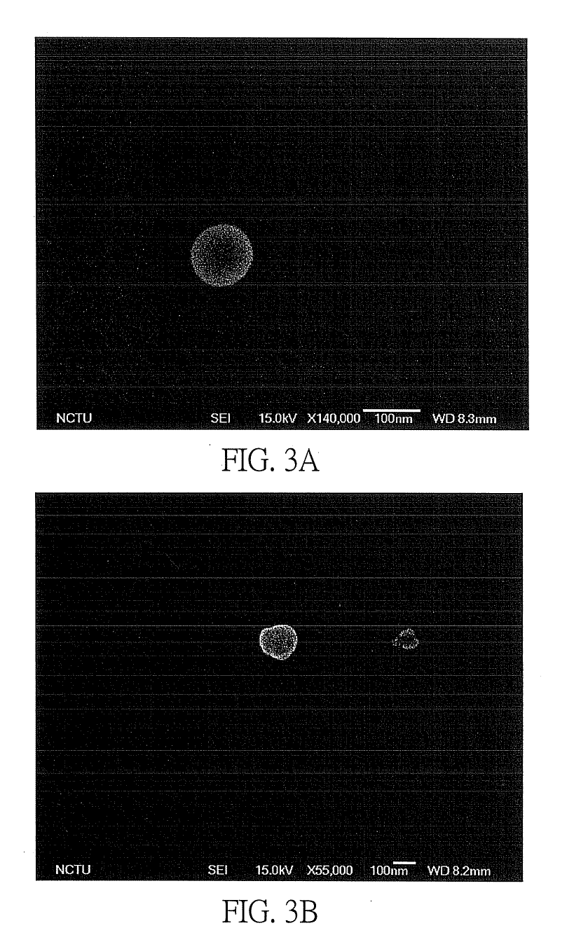

FIG. 2 shows zeta potential (the measuring device purchased from Delsa Nano C, BECKMAN COULTER) in each step of Embodiment 1. As shown in FIG. 2, the zeta potential of the original dualistic chitosan was at pH 7.5, which means that the surface has positive charges. After absorbing poly(sodium styrenesulfonate) with negative charges, the surface of the modified chitosan has negative charges. Once the second shell layer is formed, the surface of the carrier component has positive charges. Hence, it is shown that the present invention provides a carrier component having a first shell layer and a second shell layer.

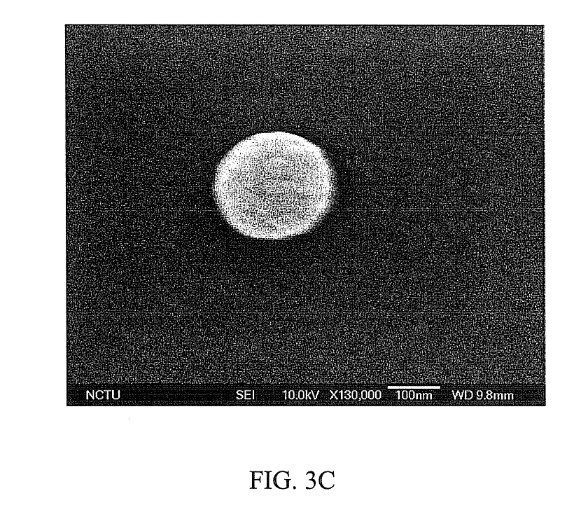

FIG. 3A to FIG. 3C are SEM images showing the product at each step in Embodiment 1. FIG. 3A shows the core body formed of the dualistic chitosan and the CPT, and the diameter of the core body is 100 to 150 nm. FIG. 3B shows the carrier component with the first shell layer formed of the CPT, the dualistic chitosan and poly(sodium styrenesulfonate), and the diameter of the carrier component is 150 to 200 nm. FIG. 3C shows the carrier component with the second shell layer formed of the CPT, the dualistic chitosan, poly(sodium styrenesulfonate) and the hydrophilic dualistic chitosan, and the diameter of the carrier component is 200 to 250 nm. The SEM images show the size of the carrier component in Embodiment 1, and also show the layer-by-layer of the carrier component.

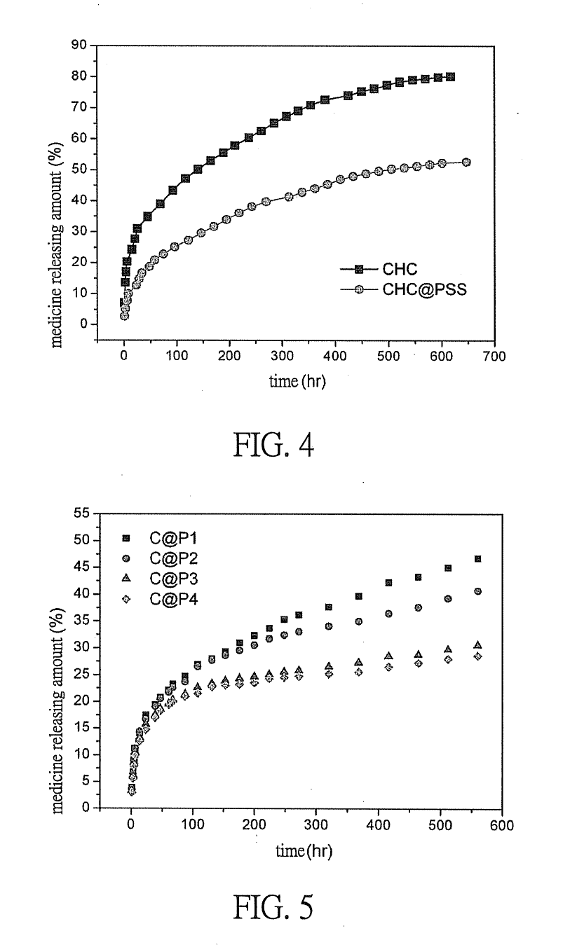

The medicine releasing amount is presented by measuring the absorption of medicine outside the carrier component in the solution with UV/visible spectrophotometer and then calculating the medicine concentration with Beer Lambert law. FIG. 4 shows the medicine releasing rates of the carrier core body (CHC) and the carrier component having the first shell layer (CHC@PSS) in Embodiment 1. As shown in FIG. 4, the carrier component of the present invention efficiently reduces the initial burst release, and also slows down the medicine releasing in the long time. The carrier component of the present invention has the layer-by-layer structure, and thus has great medicine releasing effect. Further, P1 to P4 in FIG. 5 indicate different concentration ratios of the modified dualistic chitosan to the first molecule, i.e. 1:0.3, 1:1, 1:3 and 1:5, respectively. It is noted that when the concentration of the first molecule is relatively higher, the amount of the first molecule absorbed on the carrier core body is more, and the first shell layer is thicker. As shown in FIG. 5, the medicine releasing rate of the carrier component in the present invention may be controlled by adjusting the thickness of the first shell layer.

The invention has been described using exemplary preferred embodiments. However, it is to be understood that the scope of the invention is not limited to the disclosed arrangements. The scope of the claims, therefore, should be accorded the broadest interpretation, so as to encompass all such modifications and similar arrangements.

1. A carrier component, comprising:
a carrier core body including a dispersive object and a dualistic self-assembly material for encapsulating the dispersive object, wherein the dualistic self-assembly material has an electric charge; and
a first shell layer having an electric charge opposite to the electric charge of the dualistic self-assembly material, and encapsulating the carrier core body.

2. The carrier component of claim 1, wherein the carrier core body and the first shell layer are coupled via electrostatic force.

3. The carrier component of claim 1, further comprising at least an interlayered medicine.

4. The carrier component of claim 3, wherein the interlayered medicine is disposed between the carrier core body and the first shell layer.

5. The carrier component of claim 3, wherein the interlayer medicine has an electric charge opposite to the electric charge of the dualistic self-assembly material, and is disposed in the first shell layer.

6. The carrier component of claim 1, further comprising a second shell layer having an electric charge opposite to the electric charge of the first shell layer.

7. The carrier component of claim 6, further comprising a layer-by-layer structure formed alternately from a plurality of the first shell layers and a plurality of the second shell layers.

8. The carrier component of claim 1, wherein the dualistic self-assembly material is one of an amphipathic chitosan, an amphipathic gel, a micro liposome and poly (lactic-co-glycolic acid).

9. The carrier component of claim 8, wherein the amphipathic chitosan is a chitosan derivative of formula (I): 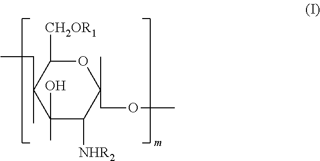
wherein R₁ is independently hydrogen, C₁-C₄alkyl, C₁-C₆carboxyl, sulfate or phosphate, R₂ is independently hydrogen, C₁-C₁₂alkyl, C₁-C₆carboxyl or C₂-C₁₂acyl, and m is an integer from 100 to 2000.

10. The carrier component of claim 8, wherein the amphipathic chitosan is a chitosan derivative of formula (II): 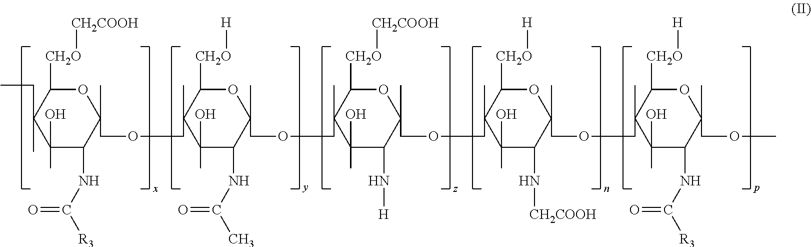
wherein R₃ is independently C₅-C₁₁alkyl, and x, y, z, n and p are integers independently from 20 to 2000.

11. The carrier component of claim 1, wherein the first shell layer is made of poly(sodium styrenesulfonate), poly(acrylic acid) (PAA), polycyclic aromatic hydrocarbon (PAH), poly (1,4-phenylene vinylene), silicon oxide or a nano gold particle.

12. The carrier component of claim 1, wherein the second shell layer is made of a hydrophilic self-assembly material.

13. The carrier component of claim 6, wherein the second shell layer is made of hydrophilic chitosan.

14. The carrier component of claim 1, wherein the carrier core body has a diameter of 100 to 150 nm.

15. The carrier component of claim 1, wherein the carrier core body has a diameter of 200 to 250 nm.

16. The carrier component of claim 1, wherein the dispersive object is a fluorescent molecule, a hydrophilic medicine, a hydrophobic medicine, a hydrophilic and hydrophobic medicine or a biological molecule.

17. The carrier component of claim 1, being a carrier component for a medicine and having an encapsulation rate of 90 to 100%.

18. The carrier component of claim 1, wherein the dispersive object is one selected from the group consisting of an anticancer drug, an anti-epilepsy drug, an SiRNA, a miRNA, a peptide, a protein, an insulin or a derivative thereof.

19. The carrier component of claim 18, further comprising at least an interlayered medicine different from the dispersive object, wherein the interlayered medicine is one selected from the group consisting of an anticancer drug, an anti-proliferative drug, an anti-hypertension drug, an anti-microbial drug, an anti-diabetes drug, an anti-fungal drug, an anti-epilepsy drug, an anti-allergy drug, an SiRNA, an miRNA, a peptide, a protein, an insulin, or a derivative thereof.

20. The carrier component of claim 1, further comprising a developing material disposed between the carrier core body and the first shell layer or disposed in the first shell layer, wherein the developing material is ferric oxide, gadolinium oxide, gadolinium complex, a platinum particle or a gold particle.

21. A method for forming a carrier component, comprising the steps of:
(A) dissolving a dispersive object in a solution containing a dualistic self-assembly material to form a carrier core body including the dispersive object and the dualistic self-assembly material encapsulating the dispersive object; and
(B) providing in the solution a first molecule having an electric charge opposite to an electric charge of the dualistic self-assembly material to form a first shell layer for encapsulating the carrier core body.

22. The method of claim 21, between the step (A) and the step (B) further comprising the step of:
(A-1) providing an interlayered medicine in the solution, wherein the interlayered medicine is disposed between the carrier core body and the first shell layer.

23. The method of claim 22, wherein the interlayered medicine has an electric charge opposite to the electric charge of the dualistic self-assembly material or has no electric charge.

24. The method of claim 22, wherein in the step (B), the solution includes an interlayered medicine, and the interlayered medicine is encapsulated by the first shell layer.

25. The method of claim 21, further comprising the step of:
(C) providing a second molecule having an electric charge opposite to the electric charge of the first shell layer to form a second shell layer.

26. The method of claim 25, between the step (B) and the step (C) further comprising the step of:
(B-1) removing the first molecule which is not adsorbed on a surface of the first shell layer.

27. The method of claim 25, after the step (C), further comprising the step of:
(C-1) cleaning the carrier component with deionized water.

28. The method of claim 25, further comprising repeating the step (B) and the step (C) at least once to form a layer-by-layer structure formed alternately of a plurality of the first shell layers and a plurality of the second shell layers.

29. The method of claim 21, wherein the dualistic self-assembly material is one of an amphipathic chitosan, an amphipathic gel, and a nano liposome.

30. The method of claim 21, wherein the first molecule is poly(sodium styrenesulfonate), poly(acrylic acid) (PAA), polycyclic aromatic hydrocarbon (PAH), poly (1,4-phenylene vinylene), silicon oxide or a nano gold particle.

31. The method of claim 21, wherein the second molecule is a hydrophilic chitosan.

32. The method of claim 21, wherein a temperature of the solution is in a range from 20 to 100° C.
